(19) United States
(12) Patent Application Publication
(10) Pub. No.: US 2022/0349001 A1
(43) Pub. Date: Nov. 3, 2022

(54) METHOD FOR SYNCHRONOUSLY SEQUENCING SENSE STRAND AND ANTISENSE STRAND OF DNA

(71) Applicant: MGI TECH CO., LTD., Shenzhen (CN)

(72) Inventors: Meihua GONG, Shenzhen (CN); Shengmao LIU, Shenzhen (CN); Chongjun XU, Shenzhen (CN); Shuang ZHOU, Shenzhen (CN); Jingjing WANG, Shenzhen (CN); Jiguang LI, Shenzhen (CN); Xiaofang WEI, Shenzhen (CN); Hui JIANG, Shenzhen (CN); Jian LIU, Shenzhen (CN)

(21) Appl. No.: 17/863,808

(22) Filed: Jul. 13, 2022

Related U.S. Application Data
(63) Continuation of application No. PCT/CN2020/072727, filed on Jan. 17, 2020.

(51) Int. Cl. C12Q 1/6869; C12Q 1/6844; C12Q 1/6804

(52) U.S. Cl. CPC C12Q 1/6869; C12Q 1/6844; C12Q 1/6804

(57) ABSTRACT

Provided is a method for synchronously sequencing a sense strand and an antisense strand of an insert DNA, including: performing two rounds of rolling circle amplification and multiple displacement amplification to obtain a DNA nano ball template including a read1 strand sequencing template and a read2 strand sequencing template; and hybridizing the read1 strand sequencing template and the read2 strand sequencing template with read1 strand sequencing primers and read2 strand sequencing primers, respectively, and simultaneously performing read1 strand sequencing and read2 strand sequencing to obtain sequences of the sense strand and the antisense strand of the insert DNA. The method of the present disclosure can perform the sequencing from both ends of the insert DNA, significantly saving the time and costs for sequencing, and increasing the sequencing throughput.

### CROSS-REFERENCE TO RELATED APPLICATIONS

The present application is a continuation of International Application No. PCT/CN2020/072727, filed on Jan. 17, 2020, the entire disclosure of which is incorporated herein by reference.

### TECHNICAL FIELD

The present application relates to the technical field of nucleic acid sequencing, and particularly, to a method for synchronously sequencing a sense strand and an antisense strand of insert DNA.

### BACKGROUND

Currently, high-throughput sequencing methods mainly include single-end sequencing and paired-end/mate-paired (PE/MP) sequencing. PE/MP sequencing is also referred to as bi-directional sequencing for determining sequences at the two ends of a long DNA fragment, and the sequences at the two ends form a “pair”. A distance between the sequences at the two ends is a length of the insert DNA fragment for sequence assembly, alignment, etc. For duplication, deletion and insertion of genome, this method is more accurate, and has a wider coverage on the genome. The difference between the paired-end sequencing and the mate-paired sequencing lies in the way of constructing the library. At present, the paired-end sequencing is dominant, as it increases the length of sequencing and can also provide a new method for structural variation analysis.

With respect to the PE/MP sequencing method, different sequencing platforms currently have different sequencing solutions, but they are generally required to sequence one strand read1 of DNA first, and thereafter sequence the complementary strand read2. For example, the PE/MP sequencing developed by Illumina, which takes the highest market share, mainly employees bridge amplification. Specifically, DNA fragmentation is first performed, adapters containing sequencing primer binding sites are added at both ends, and a template strand for a first-round sequencing of read1 is removed after the first round of sequencing is finished. Then, the complementary strand is in-situ regenerated and amplified under guidance by a Paired-End Module, so as to reach the number of templates to be used in the second round of sequencing, and the second round of sequencing by synthesis is performed on the complementary strand read2.

The existing PE/MP sequencing has the problems of lower sequencing throughput and higher sequencing cost, as the two strands of DNA need to be sequenced one after another.

### SUMMARY

The present disclosure provides a method for synchronously sequencing a sense strand and an antisense strand of insert DNA, capable of simultaneously sequencing the insert DNA from both ends of the insert DNA, and synchronously sequencing the sense and antisense strands of the insert DNA. That is, one stand read1 and a complementary strand read2 can be simultaneously sequenced, thereby greatly saving the time and costs for sequencing, and increasing the sequencing throughput.

The above are achieved by the present disclosure through the following technical solutions.

A sequencing method includes providing a sequencing template comprising a read1 strand and a read2 strand; hybridizing the sequencing template with read 1 strand sequencing primers and read2 strand sequencing primers; simultaneously performing read1 strand sequencing and read2 strand sequencing based on the read 1 strand sequencing primers and the read2 strand sequencing primers to generate read1 of the read1 strand and read2 of the read2 strand; and positioning the read1 of the read1 strand and the read2 of the read2 strand based on a signal difference between a signal of the read1 and a signal of the read2.

In preferable embodiments, the signal of the read2 is 2 times or other times the signal of the read1.

In preferable embodiments, the signal difference is caused by a difference between a copy number of the read1 strand and a copy number of the read2 strand.

In preferable embodiments, the read1 strand is a DNA nano ball formed by rolling circle amplification, and the read2 strand is formed by performing multiple displacement amplification on the DNA nano ball.

In preferable embodiments, the signal difference is realized by controlling time of the rolling circle amplification and time of the multiple displacement amplification.

A method for synchronously sequencing a sense strand and an antisense strand of insert DNA, includes: performing a first round of rolling circle amplification using a circular DNA molecule to be sequenced as an amplification template, to generate a first partial template strand; hybridizing the first partial template strand with blocked or unblocked amplification primers, and performing a second round of rolling circle amplification using the circular DNA molecule to be sequenced as an amplification template to generate a second partial template strand; selecting any one partial template strand of the first partial template strand or the second partial template strand as a read1 strand sequencing template, and generating a read2 strand sequencing template by performing multiple displacement amplification using the other partial template strand as an amplification template; and hybridizing the read1 strand sequencing template with read1 strand sequencing primers, hybridizing the read2 strand sequencing template with read2 strand sequencing primers, and simultaneously performing read1 strand sequencing and read2 strand sequencing to obtain sequences of the sense strand and the antisense strand of the insert DNA.

In preferable embodiments, the blocked amplification primers are 3′-end phosphorylated read1 strand sequencing primer; and the unblocked amplification primers are multiple-displacement-amplification primers.

In preferable embodiments, the above-described method includes the following steps: performing the first round of rolling circle amplification using a single-stranded circular DNA molecule of the DNA as the amplification template, to generate a DNA nano ball; hybridizing the first partial template strand generated by the first round of rolling circle amplification with 3′-end phosphorylated read1 strand sequencing primers; performing the second round of rolling circle amplification on the DNA nano ball to extend a 3′-end of the template strand to generate the second partial template strand, the second partial template strand being used as the read1 strand sequencing template; dephosphorylating the 3′-end phosphorylated read1 strand sequencing primers, generating the read2 strand sequencing template by performing the multiple displacement amplification, and hybridizing the read2 strand sequencing template with the read2 strand sequencing primers; hybridizing the read1 strand sequencing template with the read1 strand sequencing primers to obtain a DNA nano ball template ready to be sequenced, the DNA nano ball template being hybridized with the read1 strand sequencing primers and the read2 strand sequencing primers; and simultaneously performing the read1 strand sequencing and the read2 strand sequencing using the read1 strand sequencing primers and the read2 strand sequencing primers, to obtain the sequences of the sense strand and the antisense strand of the insert DNA.

In preferable embodiments, the above-described method includes the following steps: performing the first round of rolling circle amplification using a single-stranded circular DNA molecule as the amplification template, to generate a DNA nano ball; hybridizing the first partial template strand generated by the first round of rolling circle amplification with multiple-displacement-amplification primers; performing the second round of rolling circle amplification on the DNA nano ball to extend a 3′-end of the template strand to generate the second partial template strand, the second partial template strand being used as the read1 strand sequencing template; generating the read2 strand sequencing template by performing the multiple displacement amplification using the multiple-displacement-amplification primers, and hybridizing the read2 strand sequencing template with the read2 strand sequencing primers; hybridizing the read1 strand sequencing template with the read1 strand sequencing primers to obtain a DNA nano ball template ready to be sequenced, the DNA nano ball template being hybridized with the read1 strand sequencing primers and the read2 strand sequencing primers; and simultaneously performing the read1 strand sequencing and the read2 strand sequencing using the read1 strand sequencing primers and the read2 strand sequencing primers, to obtain the sequences of the sense strand and the antisense strand of insert DNA.

In preferable embodiments, the above-described method includes the following steps: performing the first round of rolling circle amplification using a single-stranded circular DNA molecule of the insert DNA as the amplification template, to generate a DNA nano ball; hybridizing the first partial template strand generated by the first round of rolling circle amplification with 3′-end phosphorylated read1 strand sequencing primers; performing the second round of rolling circle amplification on the insert DNA nano ball to extend a 3′-end of the template strand, wherein in the second round of rolling circle amplification, a dUTP-containing mix is used first to extend the template strand to obtain a template strand having a part containing U bases, and a dNTP mix is used then to further extend the template strand to generate the second partial template strand; hybridizing the second partial template strand with multiple-displacement-amplification primers, and performing the multiple displacement amplification to generate the read2 strand sequencing template; hybridizing the read2 strand sequencing template with the read2 strand sequencing primers, and dephosphorylating the 3′-end phosphorylated read1 strand sequencing primers to obtain a DNA nano ball template ready to be sequenced, the DNA nano ball template being hybridized with the read1 strand sequencing primers and the read2 strand sequencing primers; and simultaneously performing the read1 strand sequencing and the read2 strand sequencing using the read1 strand sequencing primers and the read2 strand sequencing primers, to obtain the sequences of the sense strand and the antisense strand of the insert DNA.

In preferable embodiments, in the above-described method, a copy number of the read1 strand sequencing template and a copy number of the read2 strand sequencing template are controlled by controlling time of the rolling circle amplification and time of the multiple displacement amplification.

In preferable embodiments, the copy number of the read1 strand sequencing template and the copy number of the read2 strand sequencing template have a difference in folds, and sequencing bases are positioned by using a signal difference caused by the difference in folds during the sequencing.

In preferable embodiments, in the above-described method, different specific antibodies with different fluorescence are added during the sequencing to specifically recognize different bases with blocking groups to acquire base signals.

In preferable embodiments, the above-described sequencing includes: firstly adding the bases with blocking groups, adding specific antibodies with different fluorescence that are capable of specifically recognizing bases with blocking groups and acquiring signals, eluting the antibodies to remove the signals, and subsequently removing the blocking groups, to enter a next round of cycle. In preferable embodiments, the above-described sequencing is performed on an MGISEQ-2000 sequencer.

In the sequencing method of the present disclosure, through two rounds of rolling circle amplification and the multiple displacement amplification, a DNA nano ball template ready to be sequenced can be obtained, and since the DNA nano ball template is hybridized with the read1 strand sequencing primers and the read2 strand sequencing primers, the read1 strand sequencing and the read2 strand sequencing can be performed simultaneously to obtain the sequences of the sense strand and the antisense strand of insert DNA. In this way, the sequencing time and sequencing cost are greatly saved, and the sequencing throughput is improved.

### BRIEF DESCRIPTION OF DRAWINGS

FIG. 1 is a principle schematic diagram of a first scheme of a method for synchronously sequencing a sense strand and an antisense strand of an insert DNA according to embodiments of the present disclosure;

FIG. 2 is a principle schematic diagram of a second scheme of a method for synchronously sequencing a sense strand and an antisense strand of an insert DNA according to embodiments of the present disclosure;

FIG. 3 is a principle schematic diagram of a third scheme of a method for synchronously sequencing a sense strand and an antisense strand of an insert DNA according to embodiments of the present disclosure;

FIG. 4 is a graph illustrating sequencing signal results of a first scheme of the method for synchronously sequencing a sense strand and an antisense strand of an insert DNA according to embodiments of the present disclosure;

FIG. 5 is a graph illustrating sequencing signal results of a second scheme of the method for synchronously sequencing a sense strand and an antisense strand of an insert DNA according to embodiments of the present disclosure; and

FIG. 6 is a graph illustrating sequencing signal results of a third scheme of the method for synchronously sequencing a sense strand and an antisense strand of an insert DNA according to embodiments of the present disclosure.

### DESCRIPTION OF EMBODIMENTS

Hereinafter, the present disclosure is described in detail by means of specific embodiments in conjunction with the accompanying drawings. In the following embodiments, many detailed descriptions are provided to facilitate the understanding of the present disclosure. However, those skilled in the art can readily realize that some of these features may be omitted under different circumstances, or may be replaced by other materials or methods.

Additionally, the features, operations, or characteristics described in the specification may be combined in any suitable manner to form various embodiments. Meanwhile, the steps or actions in the described method can also be interchanged or adjusted in terms of their orders in a manner obvious to those skilled in the art. Therefore, the order as illustrated in the specification and drawings is only for the purpose of clearly describing a certain embodiment, rather than indicating a necessary order, unless it is otherwise stated that a certain order must be followed.

The present disclosure provides a sequencing method, including: providing a sequencing template comprising a read1 strand and a read2 strand; hybridizing the sequencing template with read 1 strand sequencing primers and read2 strand sequencing primers; simultaneously performing read1 strand sequencing and read2 strand sequencing based on the read 1 strand sequencing primers and the read2 strand sequencing primers to generate read1 of the read1 strand and read2 of the read2 strand; and positioning the read1 of the read1 strand and the read2 of the read2 strand based on a signal difference between a signal of the read1 and a signal of the read2.

In some embodiments, the signal of the read2 is 2 times or other times the signal of the read1.

In some embodiments, the signal difference is caused by a difference between a copy number of the read1 strand and a copy number of the read2 strand.

In some embodiments, the read1 strand is a DNA nano ball formed by rolling circle amplification, and the read2 strand is formed by performing multiple displacement amplification on the DNA nano ball.

In some embodiments, the signal difference is realized by controlling time of the rolling circle amplification and time of the multiple displacement amplification.

In a method of the present disclosure, through two rounds of rolling circle amplification and the multiple displacement amplification, a DNA nano ball template ready to be sequenced can be obtained. The template for a read2 strand or the template for a read1 strand can be generated by controlling the time of the rolling circle amplification (RCA) and the time of the multiple displacement amplification (MDA). That is, the copy number of the read2 strand and the copy number of the read1 strand are both controllable. In some schemes, a template containing both the read2 stands and the read1 strands is generated and hybridized with the read2 strand sequencing primers and the read1 strand sequencing primers, and the sequencing bases can be positioned for the read1 strand and the read2 strand through a signal difference caused by a difference between the copy number of the read2 strand and the copy number of the read1 strand, for example, the signal of the read2 being 2 times or other times a signal of the read1, to simultaneously sequence the read1 strand and the read2 strand, thereby synchronously sequencing the sense and antisense strands of the insert DNA.

Specifically, in some embodiments of the present disclosure, a method for synchronously sequencing a sense strand and an antisense strand of an insert DNA includes the following steps: performing a first round of rolling circle amplification using a circular DNA molecule to be sequenced as an amplification template, to generate a first partial template strand; hybridizing the first partial template strand with blocked or unblocked amplification primers, and performing a second round of rolling circle amplification using the circular DNA molecule to be sequenced as an amplification template, to generate a second partial template strand; selecting one of the first partial template strand or the second partial template strand as a read1 strand sequencing template, and generating a read2 strand sequencing template by performing multiple displacement amplification using the other partial template strand as an amplification template; and hybridizing the read1 strand sequencing template with read1 strand sequencing primers, hybridizing the read2 strand sequencing template with read2 strand sequencing primers, and simultaneously performing read1 strand sequencing and read2 strand sequencing to obtain sequences of the sense strand and the antisense strand of the insert DNA.

In the present disclosure, the rolling circle amplification includes two stages, i.e., the first round of rolling circle amplification and the second round of rolling circle amplification, which generate the first partial template strand and the second partial template strand respectively. By controlling the time of the rolling circle amplification, a length of the first partial template strand and a length of the second partial template strand can be controlled, and accordingly, a copy number of the read1 strand and a copy number of the read2 strand can be controlled. The rolling circle amplification is generally carried out under the action of DNA polymerase, and the most common DNA polymerase is phi29 DNA polymerase.

In the present disclosure, either one of the first partial template strand and the second partial template strand can serve as the read1 strand sequencing template, and the other of the first partial template strand and the second partial template strand can be used as the template strand of the multiple displacement amplification to generate the read2 strand sequencing template.

In the present disclosure, subsequent to the first round of rolling circle amplification and prior to the second round of rolling circle amplification, the first partial template strand is hybridized with the blocked or unblocked amplification primers. In this way, on the one hand, the first partial template strand can be temporarily blocked, and on the other hand, these amplification primers can be used as primer sequences for the subsequent multiple displacement amplification, or as primers for the subsequent read1 strand sequencing.

In the present disclosure, these amplification primers are blocked or unblocked. The blocked amplification primer generally refers to a primer whose 3′-end is blocked by a blocking group and cannot be extended under the action of a polymerase. In an embodiment of the present disclosure, the blocked amplification primer is a 3′-end phosphorylated read1 strand sequencing primer. Since the 3′-end of the read1 strand sequencing primer is phosphorylated and cannot be extended, it remains unchanged during the second round of rolling circle amplification. Subsequent to the second round of rolling circle amplification, the 3′-end phosphorylated read1 strand sequencing primer can be dephosphorylated such that the 3′-end group of the read1 strand sequencing primer is changed into hydroxyl and thus the 3′-end can be extended under the action of a polymerase. Accordingly, the dephosphorylated read1 strand sequencing primer can be used as a primer for the subsequent multiple displacement amplification or a primer for the subsequent read1 strand sequencing, and the specific use thereof can be determined according to the design form of the technical solutions.

When the unblocked amplification primer is bound to the first partial template strand, and during the second round of rolling circle amplification, this amplification primer will initiate an extension reaction, without affecting the extension reaction of the rolling circle amplification. After the second round of rolling circle amplification, the MDA primer extension reaction can be performed under suitable reaction conditions to generate the read2 strand sequencing template. Thus, in an embodiment of the present disclosure, the unblocked amplification primer is a multiple-displacement-amplification primer.

The method for synchronously sequencing a sense strand and an antisense strand of an insert DNA according to the present disclosure can be implemented by means of various specific technical solutions. The present disclosure provides three typical detailed solutions, but the technical solutions are not limited thereto. It should be understood that on the basis of these three technical solutions, those skilled in the art can also make other suitable modified solutions.

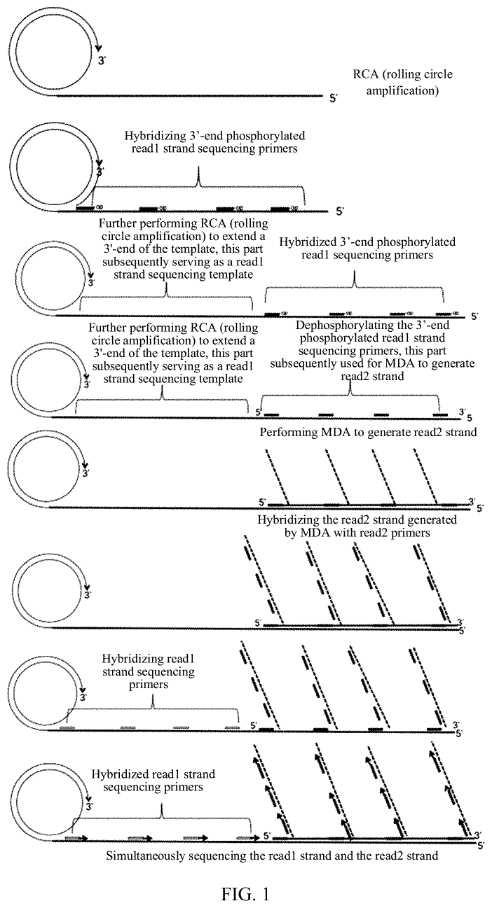

As illustrated in FIG. 1, a first detailed technical scheme of the method for synchronously sequencing a sense strand and an antisense strand of an insert DNA includes: performing a first round of rolling circle amplification (RCA) using a single-stranded circular DNA molecule of the insert DNA to be sequenced as the amplification template, to generate a DNA nano ball (DNB), without adding a stop solution; hybridizing 3′-end phosphorylated read1 strand sequencing primers on the first partial template strand generated by the first round of rolling circle amplification; then performing a second round of rolling circle amplification on the DNB to extend a 3′-end of the template strand, the extended template strand subsequently serving as a sequencing template of a read1 strand; then dephosphorylating the hybridized 3′-end phosphorylated read1 strand sequencing primers to perform multiple displacement amplification (MDA) to generate the read2 strand sequencing template, and hybridizing the read2 strand sequencing template with read2 strand sequencing primers; finally hybridizing the read1 strand sequencing primers, so that the DNA nano ball template includes the hybridized read1 strand sequencing primers as well as the read2 strand sequencing template generated and hybridized with read2 strand sequencing primers; and simultaneously performing read1 strand sequencing and read2 strand sequencing using the read1 strand sequencing primers and the read2 strand sequencing primers, to obtain the sequences of the sense strand and the antisense strand of the insert DNA. Since the time of the rolling circle amplification (RCA) and the time of the multiple displacement amplification (MDA) to generate the read2 strands are controllable, the copy number of the read2 strand and the copy number of the read1 strand can be controlled. The sequencing bases can be positioned for the read1 strand and the read2 strand simply by a signal difference brought by the copy number of the read2 strand and the copy number of the read1 strand, for example, the signal of the read2 being 2 times or other times the signal of the read1.

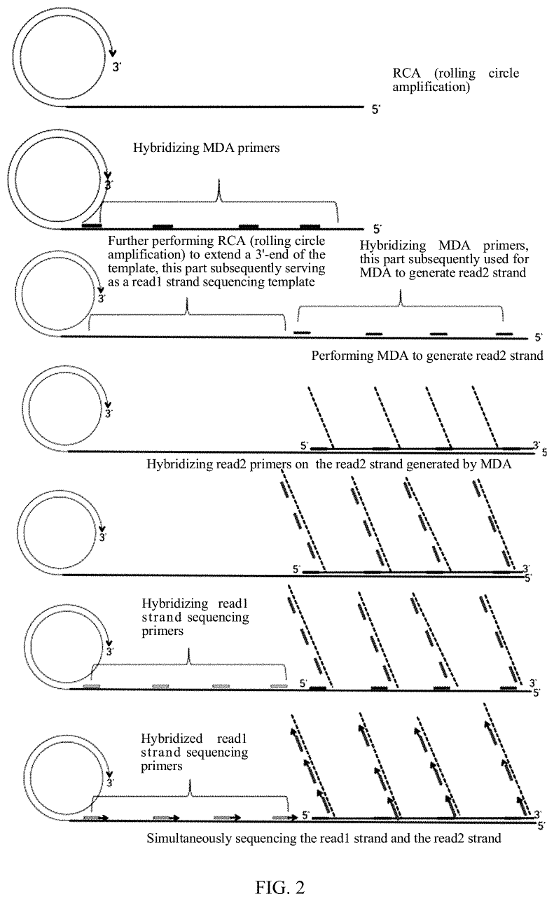

As illustrated in FIG. 2, a second detailed technical scheme of the method for synchronously sequencing a sense strand and an antisense strand of an insert DNA includes: performing a first round of rolling circle amplification (RCA) using a single-stranded circular DNA molecule as the amplification template, to generate a DNA nano ball (DNB), without adding a stop solution; hybridizing multiple-displacement-amplification (MDA) primers; then performing a second round of rolling circle amplification on the DNB to extend a 3′-end of the template strand, the extended template strand subsequently serving as a sequencing template of the read1 strand, performing multiple displacement amplification (MDA) on the part hybridized with the MDA primers, to generate the read2 strand sequencing template, and hybridizing the read2 strand sequencing template with the read2 strand sequencing primers; immediately after that, hybridizing the read1 strand sequencing template with the read1 strand sequencing primers, so that the DNB template includes the hybridized read1 strand sequencing primers as well as the read2 strand sequencing template generated and hybridized with the read2 strand sequencing primers; and finally, simultaneously performing read1 strand sequencing and read2 strand sequencing using the read1 strand sequencing primers and the read2 strand sequencing primers, to obtain the sequences of the sense strand and the antisense strand of the insert DNA. Since the time of the rolling circle amplification (RCA) and the time of the multiple displacement amplification (MDA) to generate the read2 strand template are controllable, the copy number of the read2 strand and the copy number of the read1 strand can be controlled. The sequencing bases can be positioned for the read1 strand and the read2 strand simply by a signal difference brought by the copy number of the read2 strand and the copy number of the read1 strand, for example, the signal of the read2 being 2 times or other times the signal of the read1.

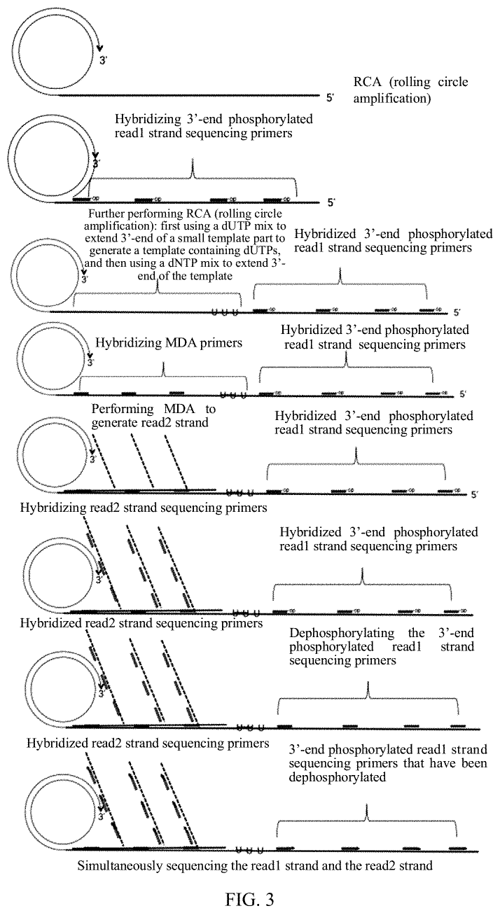

As illustrated in FIG. 3, a third detailed technical scheme of the method for synchronously sequencing a sense strand and an antisense strand of an insert DNA includes: performing a first round of rolling circle amplification (RCA) using a single-stranded circular DNA molecule as the amplification template, to generate a DNA nano ball (DNB), without adding a stop solution; hybridizing the first partial template strand generated by the first round of rolling circle amplification with 3′-end phosphorylated read1 strand sequencing primers, which can be subsequently dephosphorylated for read1 strand sequencing; then performing the second round of rolling circle amplification on the DNB in two stages to extend a 3′-end of the template strand, in which a dUTP-containing mix is used to extend the 3′-end of the template strand to obtain a template strand having a part containing U bases, the part of the template strand containing U bases cannot be recognized by phi29 DNA polymerase and thus cannot be amplified by MDA, and a dNTP mix is then used for normal extension to generate the second partial template strand which is used subsequently for MDA amplification; hybridizing the second partial template strand with the read1 strand sequencing primers, performing multiple displacement amplification (MDA) to generate the read2 strand sequencing template, hybridizing the read2 strand sequencing template with the read2 strand sequencing primers, and then dephosphorylating the 3′-end phosphorylated read1 strand sequencing primers, so that the DNB template includes the hybridized read1 strand sequencing primers as well as the read2 strand sequencing template generated and hybridized with the read2 strand sequencing primers; and finally, simultaneously performing read1 strand sequencing and read2 strand sequencing using the read1 strand sequencing primers and the read2 strand sequencing primers, to obtain the sequences of the sense strand and the antisense strand of the insert DNA. Since the time of the rolling circle amplification (RCA) and the time of the multiple displacement amplification (MDA) to generate the read2 strand are controllable, the copy number of the read2 strand and the copy number of the read1 strand can be controlled. The sequencing bases can be positioned for the read1 strand and the read2 strand simply by a signal difference brought by the copy number of the read2 strand and the copy number of the read1 strand, for example, the signal of the read2 being 2 times or other times the signal of the read1.

The methods according to the present disclosure, through appropriate modification and variation, are also applicable to synchronous sequencing of a sense strand and an antisense strand of other double-stranded DNAs or DNAs of other structures. In the variant technical solutions, different means are required to realize a method for synchronously sequence DNA sense strand and antisense strand in which both the read1 strand sequencing template and the read2 strand sequencing template are formed, and there is a certain difference between the copy number of the read2 strand and the copy number of the read1 strand so as to form a difference in terms of signal intensity between the read1 and the read2 for distinguishing and positioning the two templates.

In the method of the present disclosure, during the sequencing, different specific antibodies with different fluorescence can be added to specifically identify the different bases carrying blocking groups, so as to collect base signals. For example, in one embodiment, the bases carrying blocking groups are added firstly, then the specific antibodies with different fluorescence, which can specifically recognize the A and T bases carrying blocking groups, are added, signals are acquired, the antibodies are then eluted to remove signals, specific antibodies with different fluorescence, which can specifically recognize the C and G bases carrying blocking groups, are then added, and signals are acquired. In this way, in each round of reaction, only two types of fluorescent signals are emitted, and the read2 and the read1 have a signal difference, so as to achieve a better signal identification effect, thereby improving the sequencing quality of synchronous sequencing.

The technical solutions and effects of the present disclosure will be described in details below by way of examples. It should be understood that these examples are merely illustrative and should not be construed as limiting the present disclosure.

### Example 1

1. Instruments

MGISEQ-2000 sequencer, MGISEQ-2000 sequencing flow cell (715 nm), Mini loader, PCR machine, PCR 8-connected tubes, a set of pipettors, and High-speed centrifuge.

2. Reagents

Reagents used in the present example are listed in the following Table 1.

TABLE 1

| Reagent name | Manufacturer |
|---|---|
| DNA nano ball preparation buffer | Sigma-Aldrich |
| 3′-end phosphorylated readl strand sequencing primers | MGI |
| PNK enzyme (polynucleotide kinase) | MGI |
| Readl strand sequencing primers | MGI |
| Read2 strand sequencing primers | MGI |
| MDA primers | MGI |
| MGISEQ-2000RS high-throughput sequencing reagents | MGI |
| DNA nano ball preparation enzyme mix I | MGI |
| DNA nano ball preparation enzyme mix I | MGI |
| (dUTP-containing mix) | |
| DNA nano ball preparation enzyme mix I | MGI |
| 1XTE buffer | MGI |
| 5XSSC buffer | MGI |
| Sequencing flow cell | MGI |
| MGISEQ-2000RS high-throughput sequencer | MGI |
| DNA nano ball loading buffer I | MGI |
| DNA nano ball loading buffer II | MGI |
| Escherichia coli library | MGI |

3. Reagent Preparation:

1) Dissolution of 3′-end Phosphorylated Read1 Strand Sequencing Primers:

A 1.5 ml centrifuge tube containing the powder of the 3′-end phosphorylated read1 strand sequencing primers was placed and centrifuged on a high-speed centrifuge (Eppendorf, 5415D) for 5 minutes at a maximum speed. The primers were dissolved by 1XTE buffer to a 100 μM site-occupying primer mother solution;

2) 1 μM Working Solution of the 3′-end Phosphorylated Read1 Strand Sequencing Primers was Prepared According to Table 2 Below:

TABLE 2

| Reagent name | Volume |
|---|---|
| 100M mother solution of 3′-end phosphorylated | 100 μl |
| read 1 strand sequencing primers | |
| 5XSSC buffer | 9.9 ml |
| Total | 10 ml |

3) PNK Enzyme Reagent (for Dephosphorylation) was Prepared According to Table 3 Below:

TABLE 3

| | Final |
|---|---|
| Reagent name | concentration |
| 10U T4PNK (BGI) | 0.1U |
| 10X reaction buffer (PH5.9) | 1X |

4. Operation Steps:

1) With reference to “Instructions for MGISEQ-2000RS high-throughput sequencing reagent kit”, the preparation of DNA nano balls was performed on Escherichia coli library, 3 pieces of sequencing flow cells were prepared, and DNA nano balls were loaded on the MGISEQ-2000 sequencing flow cells (715 nm); and then according to each of the three technical solutions as described in the present disclosure, the read2 template and the read1 template were generated.

2) Three sequencing kits were prepared in accordance with the “Instructions for MGISEQ-2000RS high-throughput sequencing reagent kit”.

3) According to the “Instructions for MGISEQ-2000RS high-throughput sequencing reagent kit”, the sequencing kits and the chips were placed on MGI2000-RS sequencer, a corresponding script was selected, followed by setting SE1 and carrying out the sequencing. In order to prove the feasibility of this scheme, in this sequencing, the read2 strand sequencing primers were hybridized first, and a first cycle (cycle1) of sequencing of read2 strand was performed. After the sequencing was completed, the read2 sequencing strand was blocked, and then the read1 strand sequencing primers were hybridized to perform a first cycle (cycle1) of sequencing of read1 strand.

5. Results:

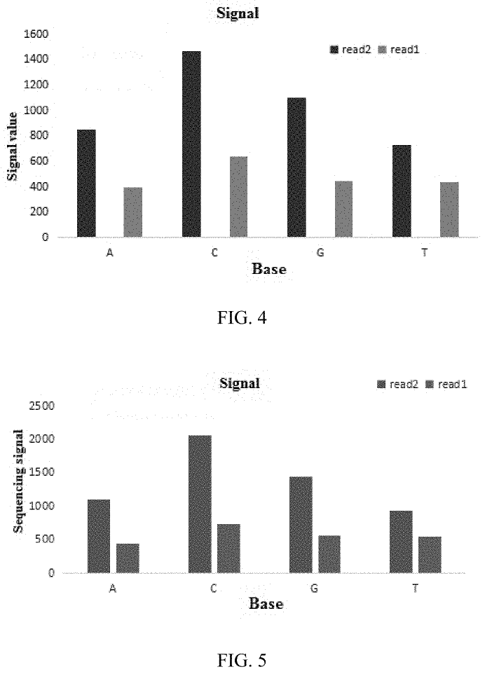

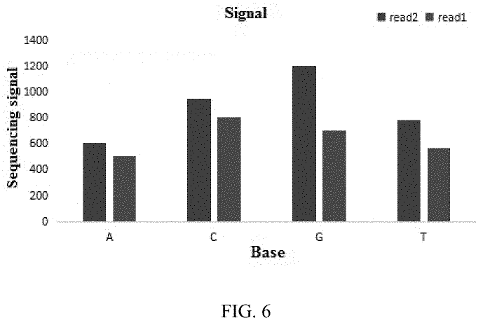

The results are shown in FIG. 4 to FIG. 6. FIG. 4 shows the sequencing signal results of the first scheme of the present disclosure, FIG. 5 shows the sequencing signal results of the second scheme of the present disclosure, and FIG. 6 shows the sequencing signal results of the third scheme of the present disclosure. The results indicate that above three schemes are all feasible, but the first scheme and the second scheme are better than the third scheme.

Specifically, the signal of read2 was about 2 times the signal of read1 in the first scheme, the signal of read2 was about 2.5 times the signal of read1 in the second scheme, while the signal of read2 was about 1.2 times the signal of read1 in the third scheme.

The specific examples as described above are intended to explain the present disclosure, only for helping to understand the present disclosure, rather than limiting the present disclosure. Those skilled in the art to which the present disclosure pertains can made several simple deductions, modifications or substitutions based on the idea of the present disclosure.

What is claimed is:

1. A sequencing method, comprising:
providing a sequencing template comprising a read1 strand and a read2 strand;
hybridizing the sequencing template with read 1 strand sequencing primers and read2 strand sequencing primers;
simultaneously performing read1 strand sequencing and read2 strand sequencing based on the read 1 strand sequencing primers and the read2 strand sequencing primers to generate read1 of the read1 strand and read2 of the read2 strand; and
positioning the read1 of the read1 strand and the read2 of the read2 strand based on a signal difference between a signal of the read1 and a signal of the read2.

2. The sequencing method according to claim 1, wherein the signal of the read2 is 2 times or other times the signal of the read1.

3. The sequencing method according to claim 1, wherein the signal difference is caused by a difference between a copy number of the read1 strand and a copy number of the read2 strand.

4. The sequencing method according to claim 1, wherein the read1 strand is a DNA nano ball formed by rolling circle amplification, and the read2 strand is formed by performing multiple displacement amplification on the DNA nano ball.

5. The sequencing method according to claim 4, wherein the signal difference is realized by controlling time of the rolling circle amplification and time of the multiple displacement amplification.

6. A method for synchronously sequencing a sense strand and an antisense strand of an insert DNA, comprising:
performing a first round of rolling circle amplification using a circular DNA molecule of the insert DNA to be sequenced as an amplification template, to generate a first partial template strand;
hybridizing the first partial template strand with blocked or unblocked amplification primers, and performing a second round of rolling circle amplification using the circular DNA molecule as an amplification template to generate a second partial template strand;
selecting any one partial template strand of the first partial template strand or the second partial template strand as a read1 strand sequencing template, and generating a read2 strand sequencing template by performing multiple displacement amplification using the other partial template strand as an amplification template; and
hybridizing the read1 strand sequencing template with read1 strand sequencing primers, hybridizing the read2 strand sequencing template with read2 strand sequencing primers, and simultaneously performing read1 strand sequencing and read2 strand sequencing to obtain sequences of the sense strand and the antisense strand of the insert DNA.

7. The method according to claim 6, wherein the blocked amplification primers are 3′-end phosphorylated read1 strand sequencing primers; and the unblocked amplification primers are multiple-displacement-amplification primers.

8. The method according to claim 6, comprising:
performing the first round of rolling circle amplification using a single-stranded circular DNA molecule of the insert DNA as the amplification template, to generate a DNA nano ball;
hybridizing the first partial template strand generated by the first round of rolling circle amplification with 3′-end phosphorylated read1 strand sequencing primers;
performing the second round of rolling circle amplification on the insert DNA nano ball to extend a 3′-end of the template strand to generate the second partial template strand, the second partial template strand being used as the read1 strand sequencing template;
dephosphorylating the 3′-end phosphorylated read1 strand sequencing primers, generating the read2 strand sequencing template by performing the multiple displacement amplification, and hybridizing the read2 strand sequencing template with the read2 strand sequencing primers;
hybridizing the read1 strand sequencing template with the read1 strand sequencing primers to obtain a DNA nano ball template ready to be sequenced, the DNA nano ball template being hybridized with the read1 strand sequencing primers and the read2 strand sequencing primers; and
simultaneously performing the read1 strand sequencing and the read2 strand sequencing using the read1 strand sequencing primers and the read2 strand sequencing primers, to obtain the sequences of the sense strand and the antisense strand of the insert DNA.

9. The method according to claim 6, comprising:
performing the first round of rolling circle amplification using a single-stranded circular DNA molecule of the insert DNA as the amplification template, to generate a DNA nano ball;
hybridizing the first partial template strand generated by the first round of rolling circle amplification with multiple-displacement-amplification primers;
performing the second round of rolling circle amplification on the insert DNA nano ball to extend a 3′-end of the template strand to generate the second partial template strand, the second partial template strand being used as the read1 strand sequencing template;
generating the read2 strand sequencing template by performing the multiple displacement amplification using the multiple-displacement-amplification primers, and hybridizing the read2 strand sequencing template with the read2 strand sequencing primers;
hybridizing the read1 strand sequencing template with the read1 strand sequencing primers to obtain a DNA nano ball template ready to be sequenced, the insert DNA nano ball template being hybridized with the read1 strand sequencing primers and the read2 strand sequencing primers; and
simultaneously performing the read1 strand sequencing and the read2 strand sequencing using the read1 strand sequencing primers and the read2 strand sequencing primers, to obtain the sequences of the sense strand and the antisense strand of the insert DNA.

10. The method according to claim 6, comprising:
performing the first round of rolling circle amplification using a single-stranded circular DNA molecule of the insert DNA as the amplification template, to generate a DNA nano ball;
hybridizing the first partial template strand generated by the first round of rolling circle amplification with 3′-end phosphorylated read1 strand sequencing primers;
performing the second round of rolling circle amplification on the DNA nano ball to extend a 3′-end of the template strand, wherein in the second round of rolling circle amplification, a dUTP-containing mix is used first to extend the template strand to obtain a template strand having a part containing U bases, and a dNTP mix is used then to further extend the template strand to generate the second partial template strand;
hybridizing the second partial template strand with multiple-displacement-amplification primers, and performing the multiple displacement amplification to generate the read2 strand sequencing template;
hybridizing the read2 strand sequencing template with the read2 strand sequencing primers, and dephosphorylating the 3′-end phosphorylated read1 strand sequencing primers to obtain a DNA nano ball template ready to be sequenced, the DNA nano ball template being hybridized with the read1 strand sequencing primers and the read2 strand sequencing primers; and
simultaneously performing the read1 strand sequencing and the read2 strand sequencing using the read1 strand sequencing primers and the read2 strand sequencing primers, to obtain the sequences of the sense strand and the antisense strand of the insert DNA.

11. The method according to claim 6, wherein a copy number of the read1 strand sequencing template and a copy number of the read2 strand sequencing template are controlled by controlling time of the rolling circle amplification and time of the multiple displacement amplification.

12. The method according to claim 11, wherein the copy number of the read1 strand sequencing template and the copy number of the read2 strand sequencing template have a difference in folds, and sequencing bases are positioned by using a signal difference caused by the difference in folds during the sequencing.

13. The method according to claim 6, wherein different specific antibodies with different fluorescence are added during the sequencing to specifically recognize different bases with blocking groups to acquire base signals.

14. The method according to claim 13, wherein the sequencing comprises:
firstly adding the bases with blocking groups,
adding specific antibodies with different fluorescence that are capable of specifically recognizing bases with blocking groups and acquiring signals, and
eluting the antibodies to remove the signals, and subsequently removing the blocking groups, to enter a next round of cycle.

15. The method according to claim 6, wherein the sequencing is performed on a MGISEQ-2000 sequencer.
